(19) United States
(12) Patent Application Publication
(10) Pub. No.: US 2007/0237498 A1
(43) Pub. Date: Oct. 11, 2007

(54) Volatile material dispenser

(75) Inventors: Thomas A. Helf, New Berlin, WI (US); David C. Belongia, Burlington, WI (US)

Correspondence Address: S.C. JOHNSON & SON, INC., 1525 HOWE STREET, RACINE, WI, 53403-2236, US

(73) Assignee: S.C. Johnson & Son, Inc., Racine, WI (US)

(21) Appl. No.: 11/396,345

(22) Filed: Mar. 31, 2006

(51) Int. Cl. A61L 9/03; A61L 9/04

(52) U.S. Cl. 392/386; 239/44

(57) ABSTRACT

A dispenser for use with an air-treating composition to be emitted includes a base having a container support and a mounting surface separate from the container support. A container is coupled to the base container support and adapted to receive the air-treating composition, and a diffuser element is coupled to the base and positioned to disperse the air-treating composition from the container into the surrounding environment. A cover is adapted for attachment to the base mounting surface. The container support remains intact when the cover is removed therefrom. The cover may include a frame for holding one of a plurality of substrates carrying different images. In addition, the base may be formed of a plastic material while the cover is formed of a ceramic material.

### FIELD OF THE DISCLOSURE

The present disclosure generally relates to apparatus for dispensing vaporizable materials, and more particularly to electronic air freshener devices.

### BACKGROUND OF THE DISCLOSURE

Volatile material dispensers are generally known for effectively combating airborne malodors in homes and in closed public buildings, such as by odor masking or destruction. Similar devices are known for dispensing insect control materials for killing or deterring the presence of unwanted pests. Various types of vapor-dispensing devices have been employed for these purposes. The most common of such devices is the aerosol container which propels minute droplets of an air freshener composition into the air. Another common type of dispensing device is a dish containing or supporting a body of gelatinous matter which, when it dries and shrinks, releases a vaporized composition into the atmosphere. Other products, such as deodorant blocks, are also used for dispensing air-treating vapors in the atmosphere by evaporation. Another group of vapor-dispensing devices utilizes a carrier material such as paper board or a porous plastic or ceramic, impregnated or coded with a vaporizable composition.

More recently, liquid or gel air-treating compositions have been placed in an enclosure, all or part of which is formed in a polymeric film through which the air-treating composition can migrate to be released as a vapor at an outer surface. The use of this type of permeable polymeric membrane controls the dispensing of air-treating vapors, and tends to eliminate great variations in rate of dispensing over the life of the product.

Wicking devices are also well known for dispensing volatile liquids into the atmosphere, such as fragrance, deodorant, disinfectant, or insecticide active agent. A typical wicking device utilizes a combination of a wick and an emanating region to dispense a volatile liquid from a liquid reservoir. Typical wicking devices are described in U.S. Pat. Nos. 1,994,932; 2,597,195; 2,802,695; 2,804,291; 3,550,853; 4,286,754; 4,413,779; 4,454,987; 4,913,350; and 5,000,383; all of which are incorporated herein by reference. Some of these wicking dispensing devices use a heat source to promote the wicking action. Heated wicking devices are described in U.S. Pat. Nos. 3,288,556; 3,431,393; 3,482,929; 3,633,881; 4,020,321; 4,968,487; 5,038,394; 5,290,546; and 5,364,027; all which are incorporated herein by reference.

Additional dispensers of the type often referred to as plug-in diffusers are described in U.S. Pat. Nos. 4,849,606; 5,937,140; and 6,478,440; which are assigned to S.C. Johnson & Son, Inc., of Racine, Wis., each of which is incorporated herein by reference.

Certain dispensers use electricity from a wall outlet to facilitate material diffusion. Such dispensers generally include a base forming a housing for a heating element and terminals extending from a rear of the base and sized for insertion into an electrical outlet. The terminals are operatively coupled to the heating element, so that the heating element generates heat when supplied with electrical power. An air treating composition, typically provided in a container, is positioned proximate the heating element so that the heat causes the composition to release vapor into the atmosphere. A faceplate may be attached to a front of the base to hide at least a portion of the base and the container holding the air treating composition. The faceplate may be formed in an aesthetically pleasing shape and/or have graphics or other visual features presented on an exterior surface thereof. To facilitate manufacture, the faceplate is typically formed of a plastic material, such as polypropylene.

In conventional diffusers, the faceplate is attached to the base such that it is either permanently affixed to the base or requires significant effort to separate the faceplate from the base. If the faceplate is separable from the base, removal of the faceplate requires at least partial disassembly of the diffuser, resulting in a diffuser that is inoperable or unsafe to use. For example, in certain known diffusers, the faceplate not only hides the base but also engages and retains the composition container in position, and therefore removal of the faceplate will require or result in displacement of the container as well. Furthermore, regardless whether it is removable, the faceplate is typically intended to be used with base over the life of the diffuser.

Other material dispensers are provided with a portable power source. U.S. Pat. No. 6,293,474 to Helf et al., and assigned to S.C. Johnson & Son, Inc., discloses a piezoelectric liquid delivery system or atomizer for production of droplets of liquid or liquid suspensions by means of a battery operated continuous action dispenser utilizing an orifice plate in communication with a piezoelectric element. The various components of the system are enclosed in a shell-like housing that provides a pleasing appearance to the consumer while permitting passage of the liquid, in spray form, from the dispenser to the atmosphere. As such, the dispenser housing is typically formed of molded plastic material to facilitate high speed fabrication.

Conventional material dispensers fail to allow a consumer to change the appearance of a cover associated with a given base. As noted above, the faceplate may present an aesthetic shape or graphic that hides at least a portion of the base and composition container. This faceplate aesthetic is static in that it does not change during the life of the diffuser. Furthermore, such covers and faceplates are typically formed of plastic material, which limits the type of cover materials that may be used, thereby limiting the different looks and feels that a given material dispenser may have.

### SUMMARY OF THE DISCLOSURE

According to certain aspects, a dispenser for use with an air-treating composition to be emitted includes a base having a container support and a mounting surface separate from the container support. A container is coupled to the base container support and adapted to receive the air-treating composition, and a diffuser element is coupled to the base and positioned to disperse the air-treating composition from the container into the surrounding environment. A cover is adapted for attachment to the base mounting surface.

According to additional aspects, a plug-in diffuser for use with a heat-activated air treating composition to be thermally diffused includes a base having a rear wall, a container support, and a mounting surface. First and second terminals extend from the base rear wall and are adapted for connection to an electrical outlet. A heating element is disposed in the base and operably coupled to the first and second terminals, the heating element being positioned proximate the receptacle. A container is adapted to receive the heat-activated air treating composition and sized for insertion into the receptacle, and a cover is removably coupled to the mounting surface and overlying at least a portion of the container, wherein the mounting surface remains in place coupled to the base when the cover is removed therefrom.

According to further aspects, a dispenser for use with an air-treating composition to be emitted includes a base formed of a plastic material and including a container support and a mounting surface, a container coupled to the base container support and adapted to receive the air-treating composition, and a diffuser element coupled to the base and positioned to disperse the air-treating composition from the container into the surrounding environment. A cover is formed of a ceramic material and includes an adapter sized to engage the base mounting surface.

### BRIEF DESCRIPTION OF THE DRAWINGS

FIG. 1 is an exploded, perspective view of a dispenser according to the present disclosure;

FIGS. 2a and 2b illustrate two examples of substrates carrying graphics that are insertable into the faceplate shown in FIG. 1; and

FIG. 3 is a perspective view of an alternative embodiment of a dispenser in accordance with the present disclosure.

FIG. 4 is a perspective view of a further alternative embodiment of a dispenser in accordance with the present disclosure.

FIG. 5 is a side elevation view of the dispenser of FIG. 4.

FIG. 6 is a perspective view of yet another embodiment of a dispenser in accordance with the present disclosure.

FIG. 7 is an exploded assembly view of the dispenser of FIG. 6.

FIG. 8 is a rear view of a cover for the dispenser of FIG. 6.

FIG. 9 is a top perspective view of a still further embodiment of a dispenser in accordance with the present disclosure.

FIG. 10 is a bottom perspective view of the dispenser of FIG. 9.

FIG. 11 is a side elevation view, in cross-section, of a control sub-assembly incorporated in the dispenser of FIG. 9.

FIG. 12 is a side elevation view, in cross-section, of an atomizer sub-assembly incorporated in the dispenser of FIG. 9.

While the present disclosure is susceptible to various modifications and alternative constructions, certain illustrative embodiments thereof have been shown in the drawings and will be described below in further detail. It should be understood, however, that there is no intention to limit the present disclosure to the specific forms disclosed, but on the contrary, the intention is to cover all modifications, alternative constructions, and equivalents falling within the spirit and scope of the present disclosure.

### DETAILED DESCRIPTION OF THE DISCLOSURE

Various material dispenser embodiments are described herein. These include: a plug-in gel, which uses a consumable packet or pouch of gel material that is heated to dispense an aromatic chemical; a plug-in scented oil, which uses a container of liquid oil having a wick that is heated to dispense an aromatic chemical; and a piezoelectric liquid delivery system, which uses a continuous action dispenser having an orifice plate in communication with a piezoelectric element to atomize and dispense liquid aromatic material. It will be appreciated, however, that the present disclosure is not limited to these specific applications, but instead may be incorporated into any diffuser structure that may benefit from the advantages disclosed herein.

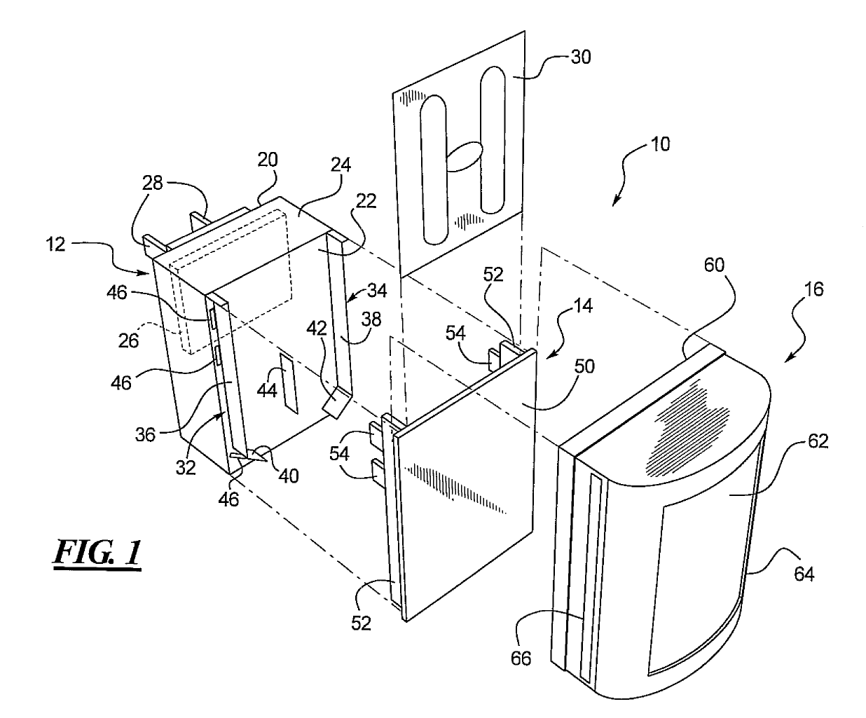

Referring now to FIG. 1, a plug-in diffuser 10 generally includes a base assembly 12, a chassis 14, and a cover in the form of a faceplate 16. The base assembly 12, chassis 14, and faceplate 16 may be attached to form an assembled diffuser suitable for receiving a container 30 holding an air treating composition, as disclosed in greater detail below. The assembled unit may then by inserted into and supported by an electrical outlet.

The base assembly 12 includes a rear wall 20, a front wall 22, and a side wall 24 that connects the front and rear walls. The rear, front, and side walls 20, 22, 24 form an enclosure in which a diffuser element, such as heating element 26, is disposed. A pair of terminals 28 project from the rear wall 20 and are sized and configured for insertion into an electrical receptacle, such as a standard wall outlet. The terminals 28 are operably coupled to the heating element so that the heating element 26 generates a desired amount of heat when the terminals 28 are inserted into a live outlet. The base assembly 12 including the heating element 26 and terminals 28 may be of any standard construction generally known in the art.

The base front wall 22 includes structure for engaging and retaining the container 30 holding a heat activatable air treating composition. In the illustrated embodiment, first and second rails 32, 34 project forwardly from the base front wall 22 and are laterally spaced from one another. The rails 32, 34 include linear, generally vertically orientated intermediate portions 36, 38 that are substantially parallel to one another and are spaced a sufficient distance to receive the entire width of the container 30 therebetween. The rails further include bottom portions 40, 42 that run inwardly and toward each other, thereby to provide a stop that engages a bottom edge of the container 30. A center support 44 also projects forwardly from the base front wall 22 and may interact with the geometry of the container 30 thereby to position the container between the rails in a desired fashion. The lateral edges of the base front wall 22 include slots 46.

The chassis 14 is coupled to the base assembly 12 and has a mounting wall 50 spaced from the base front wall 22 to form a container support in the form of a receptacle therebetween for receiving the container 30. As best shown in FIG. 1, the chassis mounting wall 50 is substantially parallel to the base front wall 22. Two side flanges 52 extend from, and are substantially perpendicular to, a rear surface of the mounting wall 50. A plurality of tabs 54 also extend from a rear surface of the mounting wall 50 and are sized for insertion into the slots 46 formed in the base front wall 22. The tabs 54 may include enlarged heads to form a snap-fit engagement when inserted through the slots 46. When the chassis 14 is coupled to the base assembly 12, the rear surface of the mounting wall 50 is spaced from the base front wall 22 by a distance sufficient to receive the thickness of the container 30. The assembled base assembly 12 and chassis 14 support the container 30 in an upright orientation and positioned sufficiently proximate the heating element 26 to effectively vaporize the air treating composition disposed in the container 30 when the heating element 26 is energized.

The base assembly 12 and chassis 14 may allow for a variety of different venting air flow paths emanating from the reservoir in which the container 30 is disposed. Direct venting paths extend out of both the top and bottom of the base/chassis sub-assembly. In addition, lateral venting paths may be provided between the rails 36, 38 of the base assembly 12 and the flanges 52 of the chassis 14. The venting allows diffusion of vaporized air treating composition to the atmosphere. The venting from the top of the base assembly provides a chimney effect so that air movement occurs across the front of the active material cartridge.

The faceplate 16 may be removably coupled to the chassis 14 to provide an aesthetically pleasing cover which hides from view at least a portion of the container 30 and base assembly 12. As best shown in FIG. 1, the faceplate 16 includes a rear flange 60 having a portion that extends inwardly from a periphery of the faceplate to engage a rear surface of the chassis mounting wall 50. The flange 60 has a depth sufficient to receive the thickness of the mounting wall 50 so that the flange 60 may slide over and be retained in position by the mounting wall 50. The faceplate 60 further includes a rear wall 62 spaced a predetermined distance from a front frame 64 to define a space therebetween. An access slot 66 may be formed in a side of the faceplate 16 and may communicate with the space between the rear wall 62 and front frame 64.

The base assembly walls 20, 22, 24, chassis 14, and faceplate 16 may be made of any material having low cost and that is easy to manufacture, such as polypropylene, nylon, and the like.

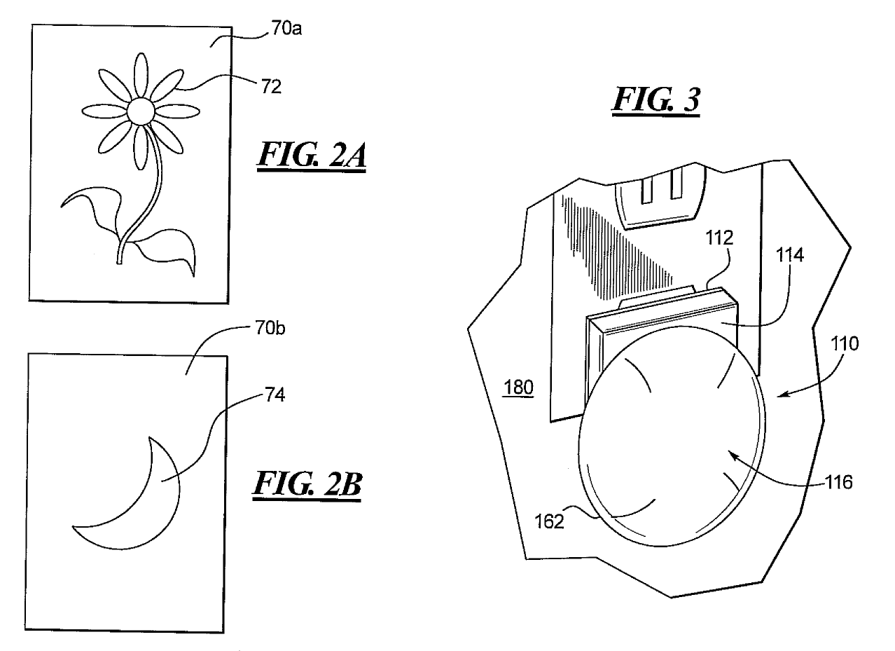

A substrate 70 carrying an image 72 is sized for insertion through the slot 66 and into the space between the rear wall 62 and front frame 64, so that the substrate image may be displayed by the faceplate 16. A set of substrates, each carrying a different image as shown in FIGS. 2a and 2b, may be provided with the diffuser 10 to allow a consumer to selectively change the appearance of the diffuser 10. For example, substrate 70a illustrated in FIG. 2a includes an image 72 of a flower, while substrate 70b illustrated in FIG. 2b includes an image 74 of a moon. While examples of two different substrates are illustrated herein, the diffuser 10 may be provided with more than two substrates, each substrate having a different image thereon. Furthermore, a substrate having a custom image, which may be provided by the consumer, can also be used with the faceplate 16.

It should be noted that the diffuser 10 continues to be functional while the faceplate 16 and/or substrate 70 is changed. The chassis 14 remains coupled to the base assembly 12 regardless whether the faceplate 16 is attached or removed. As a result, the container 30 remains in position even when the faceplate 16 is removed, thereby allowing the diffuser to function in either state.

In an alternative plug-in gel embodiment illustrated in FIG. 3, a diffuser 110 may be provided having a base assembly 112, a chassis 114, and a container (not shown) similar to the previous embodiment. A faceplate 116, however, has a display surface 162 that is unchanging or static. The appearance of the diffuser 110 may still be altered, however, by removing the faceplate 116 and replacing it with a second faceplate having a different appearance. This change is facilitated by the removable coupling of the chassis 114 and faceplate 116. As with the previous embodiment, the removable coupling may be a sliding connection between these two elements. The display surface 162 of the faceplate 116 may carry on image or other visual effect that matches the appearance of the structure located adjacent the diffuser 110. For example, the display surface 162 may have a color or texture that matches that of a wall 180. The exterior surface 162 may have a stainless steel, granite, frosted glass, or other finish.

The images carried by the different substrates may relate to different seasons, holidays, events, or other themes. In addition, the substrate may carry an image that matches the appearance of the wall or other structure carrying the electrical outlet. For example, if the adjacent wall is covered with wall paper, a piece of matching wall paper may be inserted into the space between the rear wall 162 and front frame 164, thereby to at least partially conceal or obscure the presences of the diffuser 10.

The container 30 illustrated in FIG. 1 is an active material cartridge that may be replaced when spent. Once the diffuser 10 is plugged in, the heating unit heats the air treating composition inside the active material cartridge, releasing, for example, fragrance into the atmosphere. Once the cartridge is spent, it may be removed and replaced. It should be noted that the present disclosure is not limited to use with these specifically designed refill cartridges, but instead the diffuser may also be designed as a container which itself holds a suitable active material, such as a fragrance or insect repellant, to be heated and emitted. Alternatively, a port could be provided to which separately provided containers might be attached. Preferred containers for the present disclosure comprise tray-shaped cartridge containers having a plastic laminate over the active material. The laminate includes an outer removable layer which is permeable to both liquid and vapor forms of the active material and in inner layer which is impermeable to the liquid form, but permeable to the vapor form of the active material. Upon removal of the outer layer, said active material may diffuse through the remaining layer to be released to the atmosphere. Typical of such containers are those refill units sold under the trade name GLADE® by S.C. Johnson & Son, Inc., Racine, Wis. Such cartridges are illustrated in U.S. Pat. No. 4,849,606, incorporated herein by reference.

As may be readily observed from the figures, operation of the thermal diffusers shown in FIGS. 1-3 is relatively straightforward. After insertion of an active material cartridge into the receptacle, the diffuser 10 may be plugged into an electrical outlet, thereby to power the heating element 26. When activated, heat generated by the heating element 26 is transferred by radiation and conduction to the base front wall 22. The active material cartridge, being in close proximity to the opposite side of this wall, absorbs heat energy to cause active material in the air treating composition to be heated and evaporated. The active material diffuses into the air and passes into the atmosphere through the venting paths.

The active material may be selected from a large variety of materials suitable for diffusion into the atmosphere, such as an active ingredient selected from the group consisting of fragrance, air fresheners, deodorizers, odor eliminators, odor counteractants, insecticides, insect repellants, gurble substances, medicinal substances, disinfectants, sanitizers, mood enhancers, aroma therapy compositions, and mixtures thereof. Most commonly employed active materials are fragrances and air fresheners. Preferably, the fragrance or air freshener comprises one or more volatile organic compounds which are available from perfumery suppliers such as Firmenich, Inc., Takasago, Inc., Noville, Inc., Quest Co., International Flavors & Fragrances, and Givaudan-Roure Corp. Most conventional fragrance materials are volatile essential oils. The fragrance can be a synthetically formed material, or a naturally derived oil, such as oil of bergamot, bitter orange, lemon, mandarin, caraway, cedar leaf, clove leaf, cedar wood, geranium, lavender, orange, origanum, petitgrain, white cedar, patchouli, lavandin, neroli, rose absolute, and the like.

A wide variety of chemicals are known for perfumery, such as aldehydes, ketones, esters, alcohols, terpenes, and the like. A fragrance can be relatively simple in composition, or can be a complex mixture of natural and synthetic chemical components. Synthetic types of fragrance compositions either alone or in combination with natural oils are described in U.S. Pat. Nos. 4,324,915; 4,411,829; and 4,434,306; incorporated herein by reference. Other artificial liquid fragrances include geraniol, geranyl acetate, eugenol, isoeugenol, linalool, linalyl acetate, phenethyl alcohol, methyl ethyl ketone, methylionone, isobornyl acetate, and the like.

A liquid fragrance may also be formed into a thixotropic gel by the addition of a thickening agent, such as a cellulosic material, a polymeric thickener, or a fumed silica of the type marketed under the CABOSIL trademark of Cabot Corporation. A fragrance ingredient can also be in the form of a crystalline solid, which has the ability to sublime into the vapor phase at ambient or slightly elevated temperatures. A crystalline fragrance starting material can be selected from organic compounds which include vanillin, ethyl vanillin, coumarin, tonalid, calone, heliotropene, musk xylol, cedrol, musk ketone benzophenone, raspberry ketone, methyl naphthyl ketone beta, phenyl ethyl salicylate, veltol, maltol, maple lactone, proeugenol acetate, evemyl, and the like. This type of fragrance can contribute a long term air treatment capability to an air freshener dispenser device. It is noted, however, that the present disclosure is not dependent upon the specific active material to be dispensed, but upon the novel configuration and capability of the material dispensers as disclosed herein.

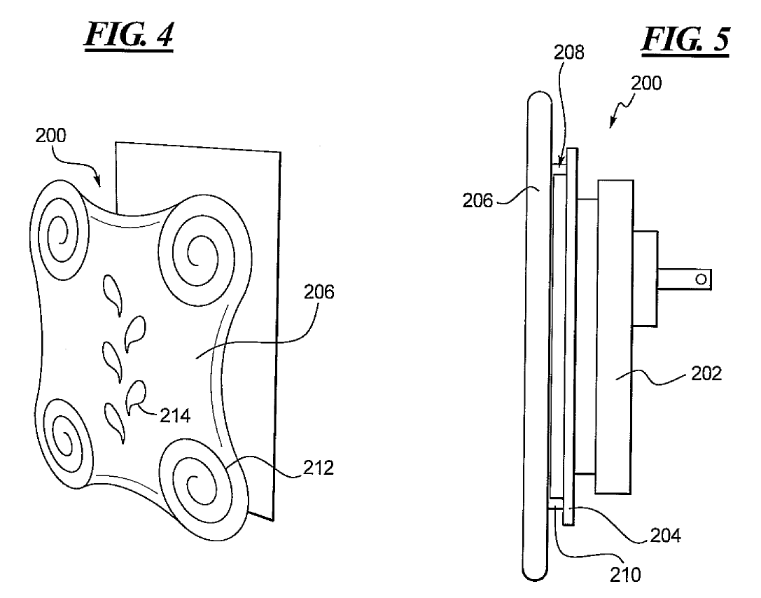

Another embodiment of a faceplate for a plug-in gel diffuser is illustrated in FIGS. 4 and 5. The plug-in gel diffuser 200 includes a base assembly 202 and chassis 204 similar to the previous embodiments. The faceplate 206 has an adapter 208 attached thereto to facilitate coupling of the faceplate 206 to the chassis 204. The adapter 208 allows the faceplate 206 to be attached either removably or permanently to the chassis 204. For example, the adapter 208 is formed with tabs 210 that fit into corresponding slots (not shown) formed in the chassis to form a removable connection therebetween. Alternatively, the adapter 208 may be glued or otherwise bonded to the chassis 204 to form a permanent attachment. In a currently preferred embodiment, the faceplate 206 is formed of a ceramic material while the adapter 208 is formed of a metal or other material that is heat-staked or otherwise bonded to the chassis 204.

The faceplate 206 may include various features for improving the aesthetics of the diffuser 200. For example, the faceplate 206 may have a stylized, attractive shape with a front surface formed with textures or designs, such as swirls 212. In addition, the faceplate 206 may include vent apertures 214 having stylized shapes, such as a tear drop shape as illustrated. When formed of ceramic material, the faceplate 206 provides a unique look, texture, and feel that is different from the typical plastic faceplates.

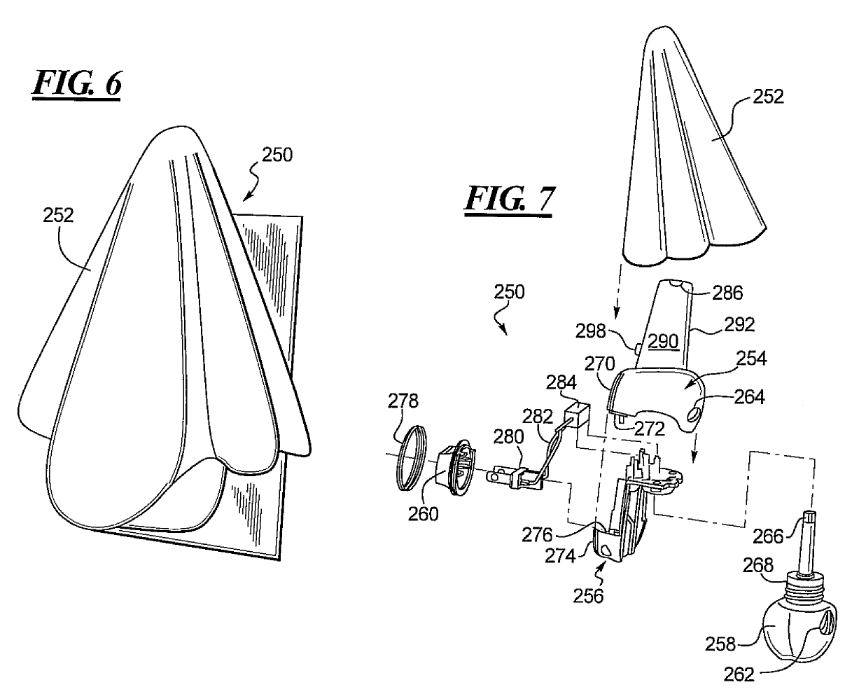

An alternative thermal-type dispenser is illustrated in FIGS. 6-8. In this embodiment, a plug-in scented oil evaporator 250 is disclosed having a cover, such as enclosure 252, attached thereto to provide an aesthetically pleasing appearance. As shown in FIG. 7, the evaporator 250 comprises a multi-piece housing, including an upper housing 254 and a lower housing 256, which form a container support for detachably retaining a bottle 258. The evaporator 250 further comprises an electrical plug assembly, including a plug deck 260, rotatably secured between the upper housing 254 and the lower housing 256.

The bottle 258 contains an evaporable substance (not shown), such as, for example, a liquid formulation including a chemical active such as an insecticide, fragrance, odor eliminator, or the like. The term “bottle” is used herein in its broadest possible sense, including any receptacle, container, pouch, etc., capable of holding a liquid formulation. A raised pattern 262 on one side of the bottle 258 is engaged by an opening 264 in a front surface of the upper housing 254 of the evaporator 250, in order to secure the bottle 258 within the evaporator 250. The front surface of the upper housing 254 is sufficiently pliant so that pulling the bottle 258 in a downward direction causes the raised pattern 262 to release from the opening 264 in the front surface of the upper housing 254, thereby enabling removal of the bottle 258 from the evaporator 250. Alternatively, a neck portion of the bottle may be designed to snap to, or screw into, the evaporator housing. Suitable refill bottles are available in a wide variety of liquid formulations from S.C. Johnson & Son, Inc., of Racine, Wis., under the GLADE® PLUGINS® and RAID® brand names.

As best shown in FIG. 8, a wick 266 is attached to the neck of the bottle 258 for drawing the liquid formulation out of the bottle 258 and toward an upper portion of the wick 266. A lower portion of the wick 266 is immersed in the liquid formulation, and the upper portion of the wick 266 protrudes above the neck of the bottle 258. Preferably, the wick 266 is positioned within the bottle 258 by a cap 268, which includes a sheath that encases the upper portion of the wick 266, except for an open area near the tip of the wick 266. Alternatively, a cap without a sheath can be utilized. Preferably, the wick 266 is about 7 mm in diameter and is constructed of ultra high molecular weight high-density polyethylene.

In the embodiment illustrated in FIGS. 6-8, the upper and lower housings 254, 256 are fastened together by heat-staking or any other suitable fastening means, including, for example, rivets, press fit, snap fit, screws, ultrasonic welding, adhesives, or the like. As best illustrated in FIG. 7, a lower back portion of the upper housing 254 forms an upper semicircular arch 270, the ends of which define an upper interface surface 272. The lower housing 256 has a corresponding inverted, semicircular arch 274, the ends of which define a lower interface surface 276. When the upper and lower housings 254, 256 are assembled, the upper and lower semicircular arches 270, 274 combine to form a ring that retains the plug deck 260. The plug deck 260 includes a groove about its circumference. When the upper and lower housings 254, 256, are assembled, the upper and lower semicircular arches 270, 274, fit into the groove in the circumference of the plug deck 260, such that the plug deck 260 is secured to the multi-piece housing in a manner that allows the plug deck 260 to rotate. The collar 278 fits over the ring formed by the upper and lower arches 270, 274, and helps to ensure that the upper and lower arches 270, 274, are held snugly together at the interface surfaces 272, 276. An electrical contact 280 protrudes through the plug deck 260 and serves the dual purpose of supplying power to the electrical components of the evaporator 250 and also supporting the evaporator 250 in a wall outlet 282 (FIG. 7). Preferably, the plug deck 260 is rotatable (as described in more detail below) in order to support the evaporator 250 in an upright position in both horizontal and vertical wall outlets.

The electrical contact 280 is electrically connected via conventional electrical conductors 282, such as wires or electrodes, to a diffuser element, such as heating device 284, for applying heat to the wick 266, when the evaporator 250 is plugged into a wall socket. The heating device 284 is positioned in the upper housing 254 near a window 286, such that it faces the tip of the wick 266 when the bottle 258 is inserted in the evaporator 250. Heating the wick 266 enhances the rate at which the liquid formulation evaporates and is diffused through the window 286 and into the surrounding environment.

Preferably, the heating device 284 is a 6 kΩ metal oxide resistor potted in a ceramic block, which is capable of handling up to at least about 5 Watts. The resistor preferably has PTC (positive temperature coefficient) characteristics, meaning that its resistance value increases slightly as the resistor heats up. A suitable resistor is available from Great Land Enterprise Co., Ltd., of Shenzhen, China, for example. Alternatively, the heating device 284 can comprise any other suitable type of heating device, such as a resistance heater, a wire-wound heater, a PTC heater, or the like.

As mentioned above, the electrical plug assembly, including the plug deck 260, is preferably rotatable in order to support the evaporator 250 in an upright position in both horizontal and vertical wall outlets. The plug deck 260 is provided with a locking mechanism, which locks the plug deck 260 in the desired one of a plurality of use positions.

The upper housing 254 includes an upwardly projecting pedestal 290 for supporting the enclosure 252. In the illustrated embodiment, the pedestal 290 has an outer mounting surface 292 having truncated, generally conic shape that tapers from a larger bottom portion to a smaller upper portion. The enclosure 252 includes an adapter, such as bracket 294, sized to slide over and engage at least a portion of the mounting surface 292. Locking means, such as an aperture 296 formed in the bracket 294 and a depressable tab projecting from the pedestal 290, may be provided to secure the enclosure 252 in place on the upper housing 254. While the illustrated enclosure 252 is removable, it may alternatively be permanently secured to the upper housing 254 by bonding, heat-staking, or other means. Additionally, the enclosure 252 may be formed of a ceramic material, in which case the bracket 294 may be formed of a metal or other material to facilitate permanent bonding with the upper housing 254, which is typically formed of a plastic material such as polypropylene. The bracket 294 may also be bonded or adhered directly to an inner surface of the enclosure 252, as illustrated in FIG. 8.

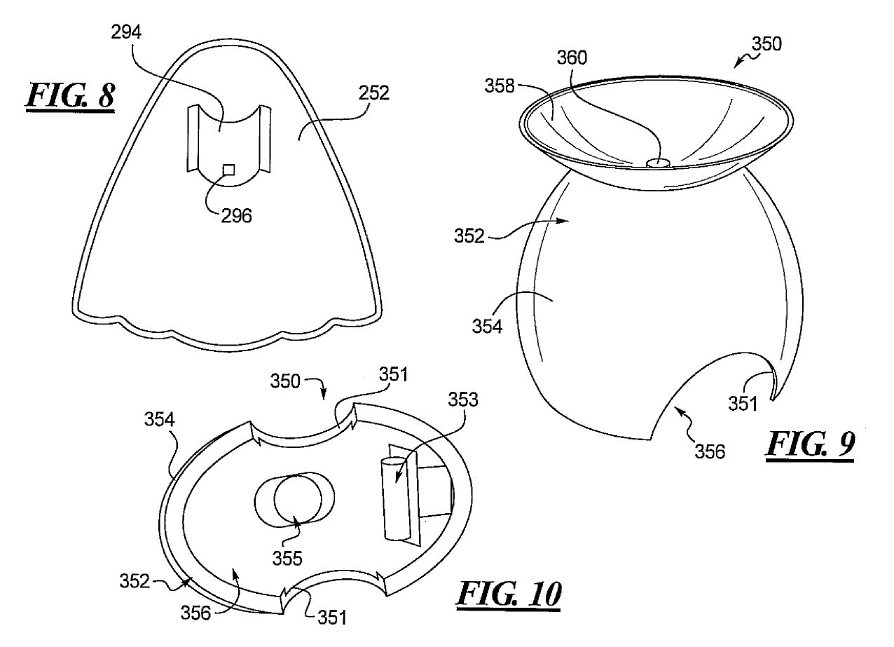

Yet another material dispenser is illustrated in FIGS. 9-12. The material dispenser includes a diffuser element in the form of an atomizer, which may diffuse an aromatic liquid without the use of heat. The illustrated atomizing device 350 includes sub-assemblies disposed within a cover, such as decorative housing 352.

More specifically, the housing includes an enclosure portion 354 having an internal surface defining a compartment 356 and a dispensing portion 358. An aperture 360 fluidly communicates between the compartment 356 and the dispensing portion 358. The housing 352 may incorporate design elements to present an attractive, aesthetically pleasing appearance. In the illustrated embodiment, the housing enclosure portion 354 has a generally semi-spherical, dome shape with a pair semi-circular openings 351 formed therein. The dispensing portion 358 is also semi-spherical and resembles a bowl. The housing 352 is preferably formed of a ceramic material to provide a unique texture, appearance, and feel not typically associated with a air treatment composition dispenser.

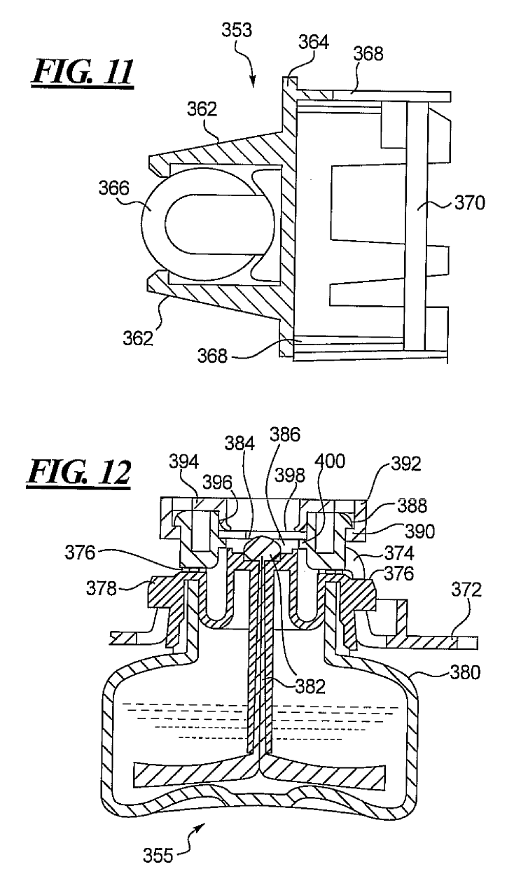

The atomizing device 350 includes a control sub-assembly 353 and an atomizing sub-assembly 355 attached to discrete portions of the housing 352 and disposed within the compartment 356, as shown in FIG. 10. The control sub-assembly, which is illustrated in greater detail in FIG. 11, includes a wall 364 coupled to a surface of the compartment 356. A pair of battery holding lugs 362 extends from the wall 364 and supports an AA battery 366. The wall 364 is formed on an opposite surface thereof with upstanding supports 368 which mount a printed circuit board 370. The printed circuit board contains circuits which are connected by leads (not shown) to the battery 366. The circuits on the printed circuit board 370 produce high frequency alternating voltages at various intervals according to the setting of a switch which is also mounted on the printed circuit board. A switch actuator (not shown) is connected to the switch to permit adjustment of the timing of these intervals.

As best shown in FIG. 12, the atomizing sub-assembly 355 includes a base wall 372 defining a mounting surface for attaching to the housing 352. The base wall 372 is formed with an upwardly extending container support 374 having a pair of diametrically opposed bayonet type slots 376 which accommodate two diametrically opposed connecting lugs 378 which extend radially outward from a liquid refill container or bottle 380. The container or bottle 380 can be removed from the atomizing device by turning it to release the lugs 378 from the bayonet slots 376. The container or bottle 380 has a wick 382 which extends out from its upper end and which conveys liquid from the container by capillary action to the bottom of an orifice plate 384.

The upper end of the container support 374 is circular in cross-section and has a center opening 386. The container support 374 is formed with an outwardly projecting snap fit bead 388 which accommodates an inwardly extending flange 390 of a retainer 392. The retainer 392 has an upper horizontal wall 394 from which flexible retainer fingers 396 extend in a downward direction. These fingers press down upon and hold an annularly shaped piezoelectric element 398 onto a shoulder 400 which is formed in the center opening 386 of the container support 374. The orifice plate 394, which contains a plurality of very small orifices or perforations, is affixed, for example by adhesive or solder, to the piezoelectric element 398 so that it extends across the bottom of the center opening of the annular piezoelectric element 398. It should be understood that the orifice plate 394 could also extend across the top of the center opening of the annular piezoelectric element 398.

The upper and lower surfaces of the piezoelectric element 398 are connected by a pair of leads (not shown) to the circuits on the printed circuit board 370. These circuits generate the alternating voltages which are applied to the actuator and which cause the actuator to expand and contract in radial directions. This in turn causes the orifice plate 384 to vibrate up and down at high frequency.

In operation of the device, the battery 366 supplies electrical power to electrical circuits on the printed circuit board 370. These circuits produce the high frequency alternating voltages which are supplied to the upper and lower sides of the piezoelectric element 398. The element 398 in turn expands and contracts and causes the orifice plate 384 to vibrate up and down as explained above. Meanwhile, the wick 382, by capillary action, draws liquid from the container 380 up to the bottom of the orifice plate 384. The up and down vibrations of the orifice plate pump this liquid through its orifices and eject it in the form of aerosolized particles from the upper surface of the plate. The ejected liquid exits from the device through the aperture 360.

### INDUSTRIAL APPLICABILITY

The volatile material dispensing system described herein incorporates features that expand the ability to provide the system with a unique appearance. In certain embodiments, a removable cover is provided which allows the appearance of the dispenser to be changed as desired. In other embodiments, the system combines the functional and aesthetic characteristics of a frame with an electric fragrance dispenser. Additionally, dispensers are disclosed which allow attachment of a ceramic cover to a plastic base, thereby providing additional options for the appearance of the dispenser.

Numerous modifications will be apparent to those skilled in the art in view of the foregoing description. Accordingly, this description is to be construed as illustrative only and is presented for the purpose of enabling those skilled in the art to make and use the invention and to teach the best mode of carrying out same. The exclusive rights to all modifications which come within the scope of the appended claims are reserved.

What is claimed is:

1. A dispenser for use with an air-treating composition to be emitted, the dispenser comprising:
a base including a container support and a mounting surface separate from the container support;
a container coupled to the base container support and adapted to receive the air-treating composition;
a diffuser element coupled to the base and positioned to disperse the air-treating composition from the container into the surrounding environment; and
a cover adapted for attachment to the base mounting surface.

2. The dispenser of claim 1, in which the diffuser element comprises a heating element.

3. The dispenser of claim 1, in which the diffuser element comprises an atomizer in fluid communication with the air-treating composition in the container.

4. The dispenser of claim 3, in which the atomizer includes a dispensing member and an actuator operably coupled to the dispensing member to selectively vibrate the dispensing member, thereby to diffuse particles of the air-treating composition.

5. The dispenser of claim 4, in which the actuator comprises a piezoelectric element.

6. The dispenser of claim 4, in which the dispensing member comprises an orifice plate.

7. The dispenser of claim 3, in which the atomizer includes a first and second sub-assemblies operably coupled to one another and independently coupled to the cover.

8. The dispenser of claim 7, in which the first sub-assembly comprises a control sub-assembly including a portable power source and a controller coupled to the portable power source.

9. The dispenser of claim 8, in which the second sub-assembly comprises an atomizing sub-assembly including a dispensing member, an actuator, and the container.

10. The dispenser of claim 1, in which the cover is permanently attached to the base mounting surface.

11. The dispenser of claim 1, in which the cover is removably attached to the base mounting surface.

12. The dispenser of claim 1, in which the base comprises a base assembly and chassis, and in which the chassis defines the mounting surface.

13. The dispenser of claim 12, in which the cover comprises a faceplate.

14. The dispenser of claim 1, in which the base comprises a pedestal, and in which the pedestal defines the mounting surface.

15. The dispenser of claim 14, in which the cover comprises an enclosure.

16. The dispenser of claim 1, in which the cover comprises a housing having an internal surface defining a compartment, and in which the base comprises a base wall, wherein the base wall is disposed within the compartment and coupled to the housing internal surface.

17. The dispenser of claim 16, in which the housing includes an enclosure portion defining the compartment, a dispensing portion, and an aperture fluidly communicating between the compartment and the dispensing portion.

18. The dispenser of claim 17, in which the diffuser element is disposed adjacent the aperture.

19. The dispenser of claim 1, in which the cover comprises a first cover adapted for releasable attachment to the base mounting surface, the dispenser further comprising a second cover adapted for releasable attachment to the base mounting surface.

20. The dispenser of claim 1, further comprising a receptacle formed in the base, first and second terminals extending from a rear wall of the base and adapted for connection to an electrical outlet, and a heating element disposed in the base, operably coupled to the first and second terminals, and positioned proximate the receptacle.

21. The dispenser of claim 20, in which the air treating composition comprises a heat-activated gel.

22. The dispenser of claim 20, in which the air treating composition comprises a scented oil and the container includes a wick sized for insertion into the receptacle.

23. The dispenser of claim 1, in which the base mounting surface comprises a plastic material and the cover comprises a ceramic material.

24. The dispenser of claim 1, in which the cover includes an adapter sized to engage the base mounting surface.

25. The dispenser of claim 24, in which the adapter comprises a metal material bonded to the cover.

26. A plug-in diffuser for use with a heat-activated air treating composition to be thermally diffused, the diffuser comprising:
a base having a rear wall, a container support, and a mounting surface;
first and second terminals extending from the base rear wall adapted for connection to an electrical outlet;
a heating element disposed in the base and operably coupled to the first and second terminals, the heating element being positioned proximate the container support;
a container coupled to the base container support and adapted to receive the heat-activated air treating composition; and
a cover removably coupled to the mounting surface and overlying at least a portion of the container, wherein the container support remains intact when the cover is removed from the mounting surface.

27. The plug-in diffuser of claim 26, in which the base further includes a front wall and the mounting surface comprises a mounting wall of a chassis, wherein the mounting wall is spaced from the base front wall to define the container support.

28. The plug-in diffuser of claim 27, in which the cover comprises a first faceplate slidably received on the chassis.

29. The plug-in diffuser of claim 26, further comprising a second faceplate adapted to be removably coupled to the chassis mounting wall, wherein the first faceplate has a front surface with a first image and the second faceplate has a front surface with a second image, and wherein a user may selectively couple one of the first and second faceplates to the chassis.

30. The plug-in diffuser of claim 26, in which the first faceplate includes a rear wall and a front frame spaced from the faceplate rear wall to define a space sufficient to receive a substrate carrying an image, the faceplate including an access slot communicating with the space and sized to receive the substrate therethrough.

31. The plug-in diffuser of claim 30, further including a plurality of substrates sized for insertion through the slot and into the faceplate, each substrate carrying a different image.

32. The plug-in diffuser of claim 26, in which the air treating composition comprises a gel.

33. The plug-in diffuser of claim 26, in which the air treating composition comprises a scented oil.

34. The plug-in diffuser of claim 26, in which the base comprises a pedestal defining an elongate receptacle, and in which the container includes an elongate wick sized for insertion into the elongate receptacle.

35. The plug-in diffuser of claim 26, in which the cover includes an adapter sized to releasably engage the mounting surface.

36. The plug-in diffuser of claim 26, in which the electrical outlet is mounted on a wall having a design feature, and in which the cover has a front surface with an image corresponding to the wall design feature.

37. A dispenser for use with an air-treating composition to be emitted, the dispenser comprising:
a base formed of a plastic material and including a container support and a mounting surface;
a container coupled to the base container support and adapted to receive the air-treating composition;
a diffuser element coupled to the base and positioned to disperse the air-treating composition from the container into the surrounding environment; and
a cover formed of a ceramic material and including an adapter sized to engage the base mounting surface.

38. The dispenser of claim 37, in which the adapter releasably engages the mounting surface.

39. The dispenser of claim 37, in which the adapter permanently engages the support mounting surface.

40. The dispenser of claim 39, in which the adapter comprises a bracket formed of a metal material.

41. The dispenser of claim 40, in which the bracket is heat-staked to the support.

42. The dispenser of claim 40, in which the bracket is bonded to the ceramic cover.
